(19) United States
(12) Patent Application Publication
(10) Pub. No.: US 2017/0342368 A1
(43) Pub. Date: Nov. 30, 2017

(54) DEVICE FOR SEPARATING REGENERATIVE AND ADULT STEM CELLS

(71) Applicant: Human Med AG, Schwerin (DE)

(72) Inventors: Inge Matthiesen, Behlendorf (DE); Konrad-Wenzel Winkler, Warin (DE)

(21) Appl. No.: 15/317,072

(22) PCT Filed: Apr. 14, 2016
(86) PCT No.: PCT/EP2016/058216

§ 371 Date: Dec. 7, 2016

(30) Foreign Application Priority Data
Jun. 10, 2015 (DE) 102015109148.8

(51) Int. Cl. C12M 1/00; C12M 1/06; C12M 1/26; C12M 1/02

(52) U.S. Cl. CPC C12M 47/04; C12M 41/18; C12M 27/02; C12M 29/04; C12M 33/04

(57) ABSTRACT

This disclosure relates to a device for separating regenerative and/or adult stem cells from a fatty tissue taken from a biological structure. In one embodiment, the device includes a container for holding a substance mixture, the substance mixture including the fatty tissue and the regenerative and/or adult stem cells, and the container including at least two chambers separated from each other by at least one membrane. The device additionally includes a substance mixture feeding facility configured to introduce the substance mixture into the container, a rinsing agent feeding facility configured to introduce a fluid into the container, a stem cell removal facility configured to remove separated stem cells from the container, a rinsing agent removal facility configured to remove a substance mixture from the container that has been at least partially depleted of regenerative and/or adult stem cells, and a specifically permeable membrane.

The present invention relates to a device for separating regenerative and/or adult stem cells from a fatty tissue removed from a biological structure.

Methods are known, for example in human medicine, for removing fatty tissue from biological structures. Fatty tissue is in a known manner suctioned out using suitable suction facilities and collected in so-called fatty tissue cell collectors.

It is known that fatty tissue obtained in this manner comprises stem cells as well as fat cells. Stem cells are body cells which can differentiate into different cell types or tissue. The stem cells present in fatty tissue are so-called regenerative and adult stem cells.

The obtaining of adult stem cells from fatty tissue is known for example from WO 2010/073808 A1. Here, the fatty tissue is mixed with a fluid containing an enzyme, and the mixture is then centrifuged in a container. This leads to a release of the stem cells from the fat cells, so that they can be collected separately.

The separation can be achieved by means of mechanical facilities such as a fluid jet, as is known for example from DE 10 2011 080 218 A1.

A device for separating adult stem cells is known from DE 10 2013 298 718 A1 which is generic for the present invention.

When such known devices for separating stem cells are used, a great deal of time is required, among other things due to a large number of preliminary and intermediate steps and for reaction processes.

It is therefore the object of the present invention to improve a generic device for separating stem cells. In particular, it should be possible with the device to minimise the amount of time required for separating the stem cells from a substance mixture removed from the patient through to the provision of re-usability of the stem cells.

The object is attained by means of the subjects of independent patent claims 1, 3, 5, 8 and 10.

The invention relates to a device for separating regenerative and/or adult stem cells from a fatty tissue taken from a biological structure, comprising a container for holding a substance mixture which comprises the fatty tissue and the regenerative and/or adult stem cells, which comprises at least two chambers which are separated from each other by at least one membrane. Further, the device comprises a substance mixture feeding facility which is designed to introduce the substance mixture into the container, and at least one rinsing agent feeding facility which is designed to introduce a fluid into the container, and a stem cell removal facility which is designed to remove the separated stem cells from the container and which is separated from the substance mixture feeding facility by the at least one membrane. The device further comprises at least one rinsing agent removal facility which is designed to remove a substance mixture which is reduced by regenerative and/or adult stem cells from the container and at least one specifically permeable membrane which is permeable for only one specific part of the respective substance mixture. According to the invention, it is provided that a first chamber of the container comprises means for separating the substance mixture and a fluid combined with the substance mixture.

A pre-treatment of the substance mixture is advantageously no longer necessary, since fluids combined with the substance mixture can be fed directly to the device. Thus, separation of the stem cells can be achieved particularly rapidly. A wide range of further advantages arises. Thus, the device according to the invention is particularly flexible. Further, the quality and yield of the stem cells are particularly good. The risks also no longer apply of a loss of substance mixture or of impurity due to upstream use of conventional devices. Further, through the device according to the invention, conventional devices for preparing the substance mixture can be used, thus reducing the financial and spatial requirements.

In a preferred embodiment of the invention, it is provided that the means for separating the substance mixture comprise a tube with a pressure compensation connection to the first chamber and a suction element connected to an underpressure source, said element being arranged in the tube.

This offers the advantage that a separation of fatty tissue, for example, and other fluids can be achieved in a particularly simple and gentle manner.

A further aspect of the present invention relates to a device for separating regenerative and/or adult stem cells from a fatty tissue taken from a biological structure, comprising a container for holding a substance mixture which comprises the fatty tissue and the regenerative and/or adult stem cells, which comprises at least two chambers which are separated from each other by at least one membrane. Further, the device comprises a substance mixture feeding facility which is designed to introduce the substance mixture into the container, and at least one rinsing agent feeding facility which is designed to introduce a fluid into the container, and a stem cell removal facility which is designed to remove the separated stem cells from the container and which is separated from the substance mixture feeding facility by the at least one membrane. The device further comprises at least one rinsing agent removal facility which is designed to remove a substance mixture which is reduced by regenerative and/or adult stem cells from the container and at least one specifically permeable membrane which is permeable for only one specific part of the respective substance mixture. According to the invention, it is provided that the at least one chamber comprises means for temperature adjustment.

This offers the advantage that biological and/or chemical processes can continuously take place in an optimal temperature range, and thus particularly rapidly. Thus, a processing time is advantageously reduced. Furthermore, the quality of the results is advantageously improved.

In a preferred embodiment of the present invention, it is provided that the means for temperature adjustment comprise a heating sleeve arranged outside of the heating chamber or a heating element arranged within the chamber and at least one temperature sensor.

This offers the advantage that the device is particularly simple and compact, and heat can be fed directly to a heat influence zone. Further, this offers the advantage that the temperature can be regulated. In a particularly preferred manner, at least one means for temperature adjustment is integrated at least in one mixing element. Thus, the heat can advantageously be brought directly in the area of the substance mixture, which at the same time can be mixed through. As a result, heat distribution is particularly homogeneous, and biological and/or chemical processes are further improved.

A further aspect of the present invention relates to a device for separating regenerative and/or adult stem cells from a fatty tissue taken from a biological structure, comprising a container for holding a substance mixture which comprises the fatty tissue and the regenerative and/or adult stem cells, which comprises at least two chambers which are separated from each other by at least one membrane. Further, the device comprises a substance mixture feeding facility which is designed to introduce the substance mixture into the container, and at least one rinsing agent feeding facility which is designed to introduce a fluid into the container, and a stem cell removal facility which is designed to remove the separated stem cells from the container and which is separated from the substance mixture feeding facility by the at least one membrane. The device further comprises at least one rinsing agent removal facility which is designed to remove a substance mixture which is reduced by regenerative and/or adult stem cells from the container and at least one specifically permeable membrane which is permeable for only one specific part of the respective substance mixture. According to the invention, it is provided that means for generating an at least partially turbulent and/or laminar flow into the substance mixture and rinsing agent are provided in the area of a membrane positioned closest to the stem cell removal device.

This offers the advantage that stem cells can no longer be added to the membrane. Thus, intermediate steps for freeing the membrane of added substances are no longer required. The processing time for separating the stem cells is advantageously reduced. The flow which can be generated can be laminar, turbulent, or partially laminar and turbulent. Due to the adaptability of flow conditions which arises, an addition to the membrane of stem cells can be particularly effectively avoided.

In a preferred embodiment of the present invention, it is provided that the means for generating an at least partially turbulent and/or laminar flow comprise a stirring element or a nozzle for generating a fluid jet. This offers the advantage that the flow can be generated in a particularly simple, targeted and gentle manner.

It is particularly preferred that the means for generating an at least partially turbulent and/or laminar flow comprise a remotely-powered electromechanical stirring element. This offers the advantage that a number of mechanical components are reduced. Further, a risk of contamination of the area with the stem cells is reduced, since no power elements or fluid conveyance elements are required in this area.

A further aspect of the present invention relates to a device for separating regenerative and/or adult stem cells from a fatty tissue taken from a biological structure, comprising a container for holding a substance mixture which comprises the fatty tissue and the regenerative and/or adult stem cells, which comprises at least two chambers which are separated from each other by at least one membrane. Further, the device comprises a substance mixture feeding facility which is designed to introduce the substance mixture into the container, and at least one rinsing agent feeding facility which is designed to introduce a fluid into the container, and a stem cell removal facility which is designed to remove the separated stem cells from the container and which is separated from the substance mixture feeding facility by the at least one membrane. The device further comprises at least one rinsing agent removal facility which is designed to remove a substance mixture which is reduced by regenerative and/or adult stem cells from the container and at least one specifically permeable membrane which is permeable for only one specific part of the respective substance mixture. According to the invention, it is provided that the rinsing agent removal facility is designed to again introduce a rinsing agent independently of the substance mixture reduced by adult stem cells and to remove it again.

This offers the advantage that downstream steps for reducing a residual collagenase concentration can be omitted, since this is directly possible in the device. The processing time for separating the stem cells through to re-usability is advantageously reduced. The stem cells can be re-used directly from out of the device, for example in the form of a return to the patient.

In a preferred embodiment of the present invention, it is provided that the device consists at least partially of sterile components. This offers the advantage that upstream steps for sterilisation can be omitted. Further, the device is thus rendered particularly safe. Devices made of sterile components offer the opportunity of designing the device as a disposable kit. The device can thus be produced entirely in a sterile location, reducing the amount of effort required at the usage site.

A further aspect of the present invention relates to a system comprising a device for separating regenerative and/or adult stem cells from a fatty tissue removed from a biological structure, and to a central control appliance. According to the invention, it is provided that the central control can be connected via a mechanical interface for the device to said device as a basis assembly, and which is capable of controlling or regulating an underpressure source and/or a temperature adjustment and/or a means for generating a flow and/or a rinsing agent conveyance facility.

This offers the advantage that all functions can be centrally adjusted, or can also be pre-defined as a program. As a result, the time required to operate the device is advantageously reduced.

The individual aspects of the invention can be advantageously combined with each other. Further preferred embodiments are described in the subclaims and in the description below.

The invention will now be explained in greater detail with reference to an exemplary embodiment and related drawings, in which:

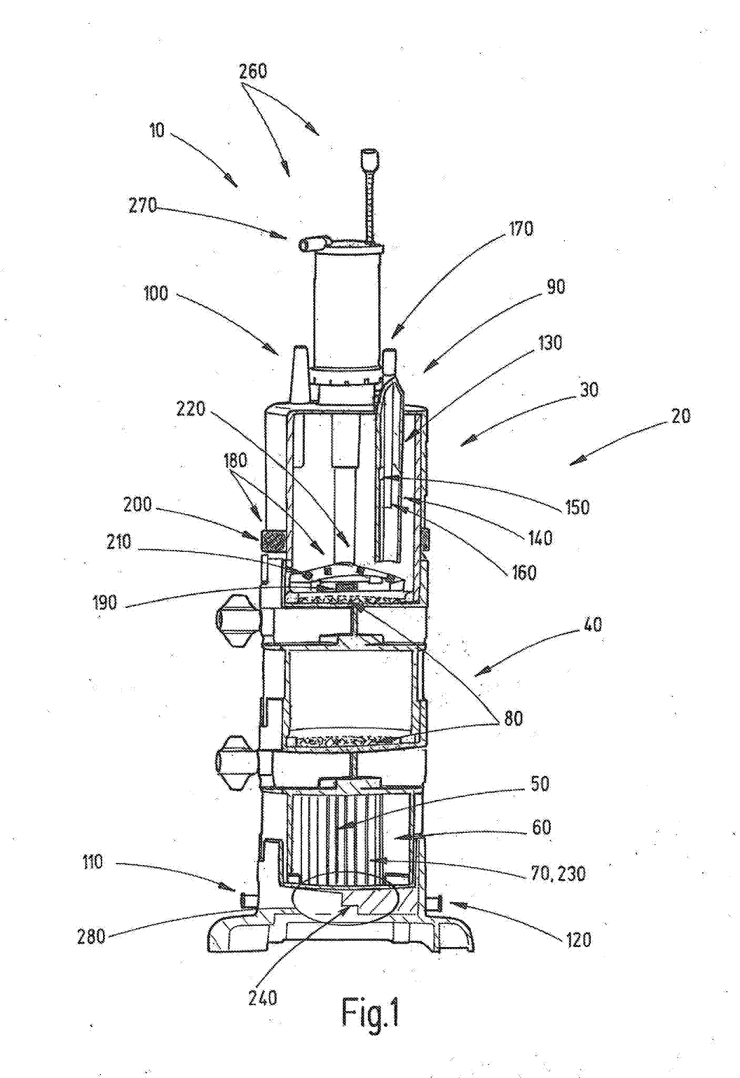

FIG. 1 shows a schematic profile view of a device according to the invention in a preferred embodiment;

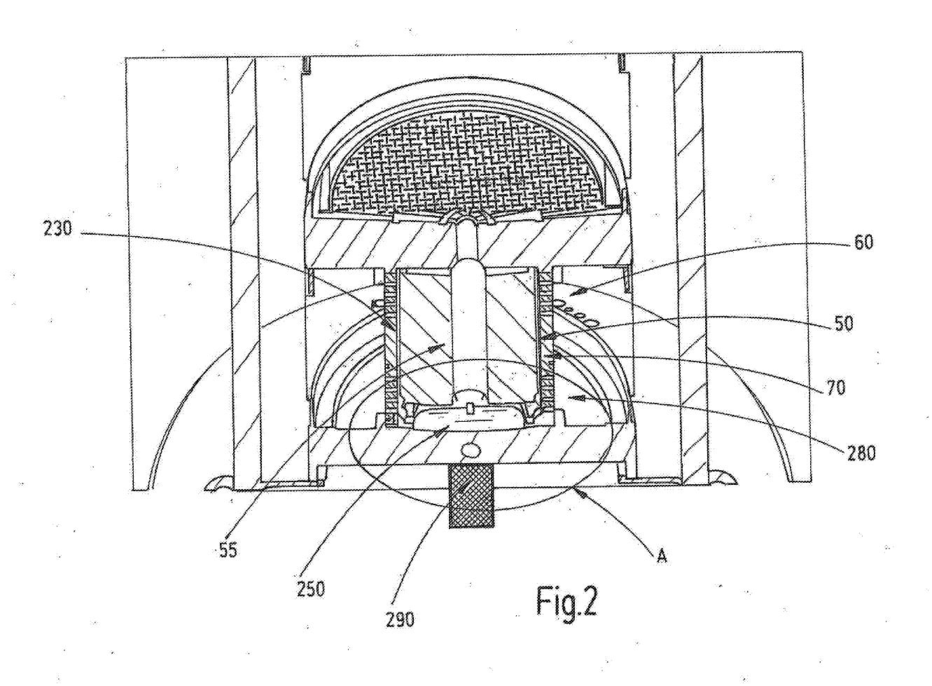

FIG. 2 shows a schematic depiction of a detailed profile view of an area of an exemplary device according to the invention with an electromechanical stirring element in a preferred embodiment, and

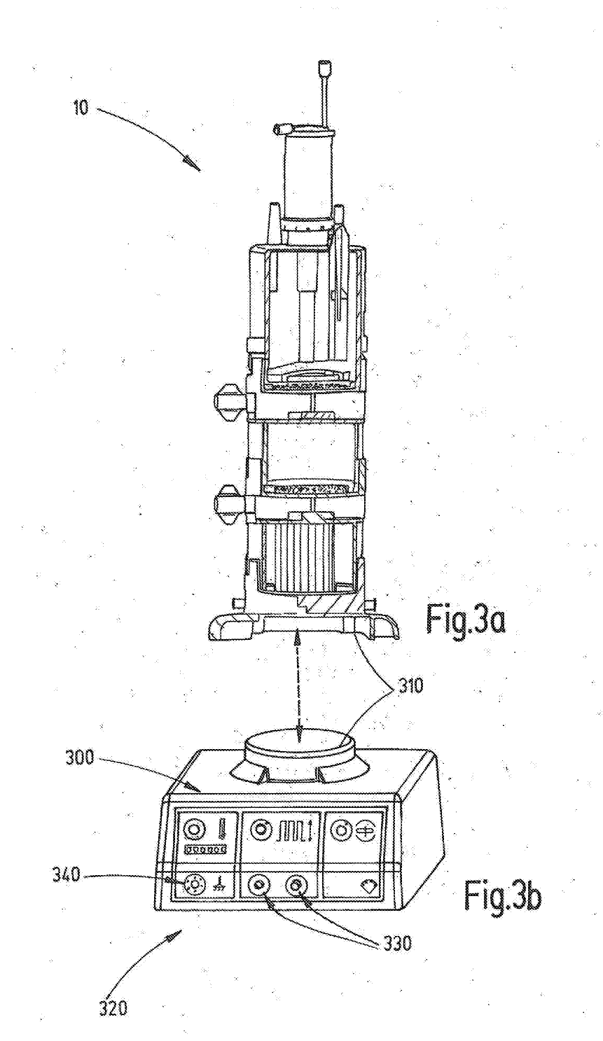

FIG. 3 shows a schematic depiction of a profile view of a system according to the invention in a preferred embodiment.

FIG. 1 shows a schematic profile view of a device according to the invention in a preferred embodiment. The device according to the invention for separating regenerative and/or adult stem cells 10 comprises a container for holding a substance mixture 20. The container 20 comprises a first chamber 30, a second chamber 40, a third chamber 50 and a fourth chamber 60. The third chamber 50 is separated from the fourth chamber 60 by a radial cylinder 70. The radial cylinder 70 comprises a filter membrane, which is designed to withhold the stem cells. The first chamber 30 is separated from the second chamber 40 and the second chamber 40 is also separated from the third chamber 50 by filter membrane 80. The first chamber 30 comprises a substance mixture feeding facility 90. The first chamber 30 further comprises a rinsing agent feeding facility 100. A schematically indicated stem cell removal facility 110 is connected to the fourth chamber 60. Further, a schematically indicated rinsing agent removal facility 120 is connected to the fourth chamber 60. The first chamber 30 of the device 10 has means for separating the substance mixture and a fluid combined with the substance mixture which can be systematically assigned to the substance mixture feeding facility 90. The means 130 comprise a tube 140 with a tube 140 with a pressure compensation connection 150 to the first chamber 30.

Further, the means 130 comprise a suction element 160 which is arranged in the tube 140 and which is connected on a suction side 170 with an underpressure source not shown here. The device 10 further comprises means for temperature adjustment 180. The means 180 comprise a heating element 190, a heating sleeve 200 and a plurality of temperature sensors 210.

The heating sleeve 200 is affixed outside on the first chamber 30. In other exemplary embodiments, further heating sleeves 200 can be affixed outside on further chambers. The heating element 190 is in this exemplary embodiment integrated into a mixing element 220. Equally, the temperature sensors 210 are integrated in the mixing element 220. In other exemplary embodiments, the arrangement and number of the heating elements 190 and temperature sensors 210 can vary. A person skilled in the art makes these adaptations depending on the process requirements. The radial cylinder 70 comprises a membrane 230 which is closest to the stem cell removal facility 110. In the area of the closest membrane 230, means are provided for generating an at least partially turbulent and/or laminar flow 240. These comprise a remotely-powered electromechanical stirring element 250, which is described in greater detail in FIG. 2. Further, the means 240 comprise an electromagnet 290 for generating an electromagnetic alternating field, which is not described further here and which is schematically indicated in FIG. 2. The rinsing agent removal facility 120 is also designed to again introduce a rinsing agent independently of the substance mixture reduced by adult stem cells and to remove it again. In the upper portion of the device 10, a connection section 260 is provided via which, for example, cables for e.g. the heating element 190 and the temperature sensors 210 can be connected and guided. Further, in this area, a connection 270 is provided for applying an underpressure. In those areas which come into contact with the substance mixture, the device 10 consists of sterile components. These components include e.g. the first chamber 30, the second chamber 40, the third chamber 50, the fourth chamber 60, the substance mixture feeding facility 90, the means for separating the substance mixture and a fluid 130 combined with the substance mixture, the mixing element 220, the filter membrane 80, the radial cylinder 70, the rinsing agent removal facility 120, the stem cell removal facility 110 and the remotely-powered electromechanical stirring element 250, not shown here. The latter is arranged in area A and is described in greater detail in FIG. 2. Area A is arranged in a lower area 280 of the radial cylinder 70. The remotely-powered electromechanical stirring element 250 has a capsular form in this exemplary embodiment. The electromagnet 290 is arranged below the remotely-powered stirring element 250.

FIG. 2 shows a schematic depiction of a detailed profile view of a partial area of a device according to the invention in a preferred embodiment. The same reference numerals apply as for FIG. 1, so that for the features referenced with these numerals, the same applies as described in FIG. 1. The partial area described is a partial area described in FIG. 1 for separating regenerative and adult stem cells 10. The partial area comprises e.g. a third chamber 50 and a fourth chamber 60, an area A and a remotely-powered electromechanical stirring element 250 and the electromagnet 290. A volume of the third chamber 50 is largely filled by a core element for volume reduction 55, so that only a small gap remains free between the core element for volume reduction 55 and the closest membrane 230. The electromagnet 290 is arranged below the remotely-powered electromechanical stirring element 250 and is designed to generate an electric alternating field in order to move the remotely-powered electromechanical stirring element 250. Thus, within the radial cylinder 70, a partially turbulent and partially laminar flow can be set. In other exemplary embodiments, purely turbulent or purely laminar flows can also be generated, e.g. with a nozzle.

FIG. 3 shows a schematic depiction of a profile view of a system according to the invention in a preferred embodiment. The same reference numerals apply as for FIG. 1, so that for the features referenced with these numerals, the same applies as described in FIG. 1. FIG. 3 shows the device for separating regenerative and adult stem cells 10 in connection with a central control appliance 300. The device for separating regenerative and adult stem cells 10 is described in FIG. 3a. The central control appliance 300 is described in FIG. 3b. The central control appliance 300 and the device 10 have a mechanical interface 310, so that the device 10 can be arranged on the central control appliance 300. The latter has a plurality of connection and operating elements 320. The central control appliance 300 is designed to regulate an underpressure source, a temperature adjustment, means for generating a flow in the form of a mechanical stirring element 250 remotely powered by an electromagnet, and a rinsing agent conveyance facility. The central control appliance 300 has e.g. connections for media 330 and connections for power and signals 340.

### LIST OF REFERENCE NUMERALS

10 Device for separating regenerative and/or adult stem cells
20 Container for holding a substance mixture
30 First chamber
40 Second chamber
50 Third chamber
55 Core element for volume reduction
60 Fourth chamber
70 Radial cylinder
80 Filter membranes
90 Substance mixture feeding facility
100 Rinsing agent feeding facility
110 Stem cell removal facility
120 Rinsing agent removal facility
130 Means for separating the substance mixture and a fluid combined with the substance mixture
140 Tubes
150 Pressure compensation connection
160 Suction element
170 Suction side
180 Means for temperature adjustment
190 Heating element
200 Heating sleeve
210 Temperature sensors
220 Mixing element
230 Closest membrane
240 Means for generating an at least partially turbulent and/or laminar flow
250 Remotely-powered electromechanical stirring element
260 Connection section
270 Connection
280 Lower area
290 Electromagnet
300 Central control appliance
310 Mechanical interface
320 Connection and operating elements
330 Connections for media
340 Connections for power and signals
A Area

1. A device for separating one or more of regenerative cells, adult stem cells, or a combination thereof from a fatty tissue taken from a biological structure, comprising:
a container for holding a substance mixture, the substance mixture comprising the fatty tissue and the one or more of regenerative cells, adult stem cells or a combination thereof, wherein the container comprises at least two chambers separated from each other by at least one membrane;
a substance mixture feeding facility configured to introduce the substance mixture into the container;
at least one rinsing agent feeding facility configured to introduce a fluid into the container;
a stem cell removal facility configured to remove the one or more of regenerative cells, adult stem cells, or a combination thereof from the container, and wherein the stem cell removal facility is separated from the substance mixture feeding facility by the at least one membrane;
at least one rinsing agent removal facility configured to remove the substance mixture from the container, the substance mixture being reduced by at least a portion of the one or more of regenerative cells, adult stem cells, or a combination thereof; and
at least one specifically permeable membrane, wherein the specifically permeable membrane is permeable for a specific part of the substance mixture.

2. The device according to claim 11, wherein the means for separating the substance mixture comprise a tube with a pressure compensation connection to a first chamber and a suction element connected to an underpressure source, said suction element being arranged in the tube.

3. The device according to claim 1, wherein at least one chamber of the at least two chambers comprises means for temperature adjustment.

4. The device according to claim 3, wherein the means for temperature adjustment comprise at least one temperature sensor and at least one of a heating sleeve arranged outside of the heating chamber or a heating element arranged within the chamber.

5. The device according to claim 1, further comprising a means for generating one or more of an at least partially turbulent flow and an at least partially laminar flow into the substance mixture and rinsing agent, wherein said means for generating one or more of an at least partially turbulent flow and an at least partially laminar flow are provided in an area of a membrane positioned closest to the stem cell removal device.

6. The device according to claim 5, wherein said means for generating one or more of an at least partially turbulent flow and an at least partially laminar flow comprise a stirring element or a nozzle for generating a fluid jet.

7. The device according to claim 5, wherein said means for generating one or more of an at least partially turbulent flow and an at least partially laminar flow comprise a remotely-powered electromechanical stirring element.

8. The device according to claim 1, wherein the rinsing agent removal facility is configured to again introduce a rinsing agent independently of the substance mixture that has been reduced by the at least a portion of the one or more of regenerative cells, adult stem cells, or a combination thereof and to remove it again.

9. The device according to claim 1, wherein the device at least partially consists of sterile components.

10. A system for separating one or more of regenerative cells, adult stem cells, or a combination thereof from a fatty tissue taken from a biological structure, comprising:
the device according to claim 1; and
a central control appliance,
wherein the central control appliance is connected via a mechanical interface for the device to said device as a basis assembly, and
wherein the central control appliance is configured to control or regulate one or more of an underpressure source, a temperature adjustment, a means for generating a flow, a rinsing agent conveyance facility, or combinations thereof.

11. The device according to claim 1, wherein a first chamber of the at least two chambers of the container comprises means for separating the substance mixture and a fluid combined with the substance mixture.
